(19) United States
(12) Patent Application Publication
(10) Pub. No.: US 2005/0020499 A1
(43) Pub. Date: Jan. 27, 2005

(54) Methods of attenuating cocaine seeking behavior employing glial cell-derived neurotrophic factor (GDNF) and pharmaceutical compositions and articles of manufacture suited for use in practice of the method

(75) Inventor: Gal Yadid, Shoham (IL)

Correspondence Address: DR. MARK FRIEDMAN LTD.;c/o Bill Polkinghorn, Discovery Dispatch, 9003 Florin Way, Upper Marlboro, MD, 20772, US

(73) Assignee: Bar Ilan University

(21) Appl. No.: 10/853,098

(22) Filed: May 26, 2004

Related U.S. Application Data
(60) Provisional application No. 60/473,126, filed on May 27, 2003.

(51) Int. Cl. A61K 38/18

(52) U.S. Cl. 514/12

(57) ABSTRACT

A method of attenuating cocaine-seeking behavior in a subject. The method includes administering a physiologically effective amount of glial cell-derived neurotrophic factor (GDNF) into a selected region of the brain. Preferaby a controlled release mechanism is employed. GDNF as a pharmaceutical composition, preferably supplied as an article of manufacture including instructions for use in attenuating cocaine-seeking behavior, is also disclosed. The claimed invention includes, but is not limited to, a physiologically effective dose in the range of one to twenty micrograms/day.

This application claims priority from U.S. Patent Application 60/473,126 filed on May 27, 2003 and currently pending.

### FIELD AND BACKGROUND OF THE INVENTION

The present invention relates to methods of attenuating cocaine-seeking behavior employing glial cell-derived neurotrophic factor (GDNF) and pharmaceutical compositions and articles of manufacture suited for use in practice of the method and, more particularly, to intra-brain delivery of GDNF in a controlled fashion to a cocaine habituated subject.

Cocaine's recent notoriety belies the fact that the drug has been used as a stimulant by people for thousands of years. Cocaine's highly addictive nature and addicts' willingness to pay a high price for the drug have propelled it into the public eye. Cocaine abuse and addiction continues to be a problem that plagues the world. In 1997, an estimated 1.5 million Americans age 12 and older were chronic cocaine user (Substance Abuse and Mental Health Services Administration, Preliminary results from the 1997 National household survey on Drug Abuse. SAMSA, 1998.). By the year 2000, the number of cocaine users had increased to 2.7 million (ONDCP Drug Policy Information Clearinghouse Fact Sheet, Executive Office of the President Office of National Drug Control Policy, 2001) Today more is known about where and how cocaine acts in the brain, including how the drug produces its pleasurable effects and why it is so addictive. Through the use of sophisticated technology, scientists can actually see the dynamic changes that occur in the brain as an individual takes the drug. Because these types of studies pinpoint specific brain regions, they are critical to identifying targets for developing medications to treat cocaine addiction. Cocaine abusers, especially those who inject, are at increased risk for contracting such infectious diseases as human immunodeficiency virus (HIV/AIDS) and hepatitis. The full extent of the effects of prenatal drug exposure on a child is not completely known, but many scientific studies have documented that babies born to mothers who abuse cocaine during pregnancy are often prematurely delivered, have low birth weights and smaller head circumferences, and are often shorter in length.

In its pure form, cocaine is a white crystalline powder extracted from the leaves of the South American coca plant. Cocaine users most often inhale the powder sharply through the nose, where it is quickly absorbed into the bloodstream. But it also can be heated into a liquid and its fumes inhaled through a pipe in a method called “freebasing”. Freebasing is also a common method of using a form of cocaine called “crack”. Crack resembles small pieces of rock and is often called “rock” on the street. Freebasing is an especially dangerous means of abusing cocaine because of the high concentrations of cocaine it introduces into the bloodstream. These high doses can overtax the cardiovascular system. Reports of sudden death while freebasing are not uncommon. Cocaine is highly addictive, especially in the crack form. In studies, animals addicted to cocaine preferred the drug to food, even when it meant they would starve. Many users report being “hooked” after only one use. The addiction is both psychological and physical. Treatment can be costly and the craving for cocaine may persist for long periods of time. For purposes of this specification and the accompanying claims, the phrase “cocaine-seeking behavior” refers to an expressed desire for cocaine as a powder (whether injected or inhaled), freebase cocaine or crack cocaine (a.k.a. rock cocaine).

Unfortunately, despite the long tenure of cocaine as a drug of abuse, no medications are currently available to treat cocaine addiction specifically. Consequently, NIDA is aggressively pursuing the identification and testing of new cocaine treatment medications. Several newly emerging compounds are being investigated to assess their safety and efficacy in treating cocaine addiction.

For example, one of the most promising anti-cocaine drug medications to date, selegeline, was taken into multi-site phase III clinical trials in 1999. These trials will evaluate two innovative routes of selegeline administration: a transdermal patch and a time-released pill, to determine which is most beneficial.

Disulfiram, a medication that has been used to treat alcoholism, has also been shown, in clinical studies, to be effective in reducing cocaine abuse. Because of mood changes experienced during the early stages of cocaine abstinence, antidepressant drugs have been shown to be beneficial. In addition to the problems of treating addiction, cocaine overdose results in many deaths every year, and medical treatments are being developed to deal with the acute emergencies resulting from excessive cocaine abuse.

It is significant to note that many cocaine addicts will revert to cocaine seeking behavior even after their physical addiction has been overcome.

The effects of cocaine on the brain may be viewed as a form of neuronal plasticity (Sklair-Tavron et al., 1996, Nestler et al, 1993). Neurotrophic factors such as GDNF (glial cell-derived neurotrophic factor) and BDNF (brain-derived neurotrophic factor) have been found to be involved in many forms of plasticity within the adult brain, including exposure to drugs of abuse (Smith et al., 1995).

GDNF is a glycosylated, disulfide-bonded homodimer, a member of the transforming growth factor (TGF-β) family. GDNF is found in various organs, including astrocyte cells in the brain (Suter-Crazzolara and Unisker, 1994, Schaar et al., 1993).

GDNF has been shown to potently promote the survival and morphological differentiation of embryonic DA neurons in vitro (Lin et al., 1993; Beck et al., 1995); it greatly enhances the survival of mature midbrain neurons in vivo following treatment with dopamine neurotoxins (Kearns and Gash, 1995), and protects animals from the behavioral deficits caused by such lesions (Gash et al., (1996) Nature 380:252-252).

Recent studies have shown that GDNF is involved in the chronic effects of cocaine on the brain. Prenatal cocaine exposure has been found to reduce striatal GDNF production in rat fetuses (Lipton et al., 1999). This effect was related to the teratogenic properties of cocaine, since cocaine is known to cause developmental alterations to dopaminergic systems, which may be affected by the reduction in GDNF levels. Binding of GDNF to the receptor complex GFR-α1-Ret causes the phosphorylation and subsequent activation of Ret, which mediates the physiological actions of GDNF (Treanor et al., 1996, Trupp et al., 1996). Messer et al. (2000) have found a substantial decrease in the levels of tyrosine-phosphorylated Ret in the VTA Thus, it has been hypothesized that some of cocaine's long-term effects on the brain are attained via cocaine intervention in endogenous GDNF signal pathways. However, Messer specifically teaches that chronic drug exposure does not alter expression levels of GDNF. This teaching implies that administration of GDNF is unlikely to have any impact on cocaine seeking behavior. Thus it can be said that chronic exposure to substances of abuse induces changes in neuron morphology concurrently with biochemical and behavioral adaptations in the dopaminergic (DAergic) system (Koob et al., 1998; Nestler & Aghajanian, 1997). Neurotrophic factors such as glial cell line-derived neurotrophic factor (GDNF) and brain-derived neurotrophic factor are also implicated in inducing neuronal plasticity (Pierce & Bari, 2001) in addition to growth and development (Nagtegaal et al., 1998). Intra-ventral tegmental area (VTA) infusion of neurotrophic factors alters drug-induced morphological and physiological effects (Berhow et al., 1995; Sklair-Tavron et al., 1996). Yet there are contrasting effects of GDNF and brain-derived neurotrophic factor on the behavioral responses to abused substances. Brain-derived neurotrophic factor dramatically augments (Horger et al, 1999), while GDNF decreases the response to cocaine administration (Messer et al., 2000).

Chronic exposure to cocaine alters GDNF production and signaling levels. Chronic cocaine exposure decreases VTA levels of tyrosine-phosphorylated Ret (Messer et al., 2000), which mediates GDNF's physiological actions (Airaksinen et al., 1999). Furthermore, prenatal cocaine exposure reduces striatal GDNF production in rat fetuses, which may impair DAergic neuronal differentiation and decrease DAergic neuron levels (Lipton et al., 1999).

Cell transplantation may be used to deliver peptide-based therapeutics such as neurotrophic factors, overcoming such difficulties as short half-lives, chemical instability, low oral bioavailability and poor blood-brain barrier penetration (Tresco et al., 2000). This technique repairs neurodegenerative and neuroplastic damage and improves neurotoxin-induced behavioral deficits (Gash et al., 1996; Yadid et al., 1999). Astrocytes, especially fetal (Sullivan et al., 1998), can be successfully transplanted into the central nervous system without tumor formation (Blakemore & Franklin, 1991) and integrate well into brain parenchyma (Tomatore et al., 1996). The immortalized, but not malignant, human astrocyte-like cell line (simian virus-40 glial —SVG) secretes GDNF tonically and following DAergic stimulation (Kinor et al., 2001). In a rat model of Parkinson's disease, SVG cells grafted into the brain remained in the tract at the transplantation site (Tomatore et al., 1996) and in primate brain, were not rejected up to nine months post-transplantation (Tomatore et al., 1993). Thus, SVG cells are potential tools for the introduction of GDNF into the brain and may be a novel approach to provide protection against biochemical and behavioral damage caused by abused substances. Other methods for treating cocaine addiction have been examined, but with limited efficacy (Carroll et al., 1999).

Magnetic nanoparticles are spheric polymeric particles made of natural or artificial polymers, ranging in size between 10-1000 nm. Due to their spherical shape, high surface area and magnetic properties, these particles have a wide range of potential applications (Berry CC, Curtis A Functionalisation of magnetic nanoparticles for applications in biomedics. Center for Cell Engineering, Institute of Biomedical and Life Sciences, University of Glasgow, Glasgow, UK. From Journal of Physics D: Applied Physics 2003 36(13) R198-R206). These particles can bind to various drugs and facilitate their delivery into the brain. Drugs bound to nanoparticles may be targeted to the brain, where they enhance the effectiveness of the drug. In addition, binding of therapeutic drugs to nanoparticles may have the potential to provide the drug with long-term protection from enzymatic degradation and other harmful environmental factors. This could serve to increase drug efficacy by increasing the time that a drug remains active (Margel S, Sturchak S and Tennebaum T, Biological glues based on thrombin conjugated nanoparticles; Brigger I, Dubemet C, Couvreur P. Nanoparticles in cancer therapy and diagnosis. Adv Drug Deliv Rev. 2002 Sep. 13;54(5):631-51; Allen T M, Cullis P R. Drug delivery systems: entering the mainstream. Science. 2004 Mar. 19; 303(5665): 1818-22.).

There is thus a widely recognized need for, and it would be highly advantageous to have, methods of attenuating cocaine seeking behavior employing glial cell-derived neurotrophic factor (GDNF) and pharmaceutical compositions and articles of manufacture suited for use in practice of the method devoid of the above limitation.

### SUMMARY OF THE INVENTION

According to one aspect of the present invention there is provided a method of attenuating cocaine-seeking behavior in a subject. The method includes administering into a selected region of a brain of the subject a physiologically effective amount of glial cell-derived neurotrophic factor (GDNF) by means of a controlled release mechanism.

According to another aspect of the present invention there is provided a pharmaceutical composition. The pharmaceutical composition includes as an active ingredient a physiologically effective amount of GDNF and physiologically acceptable carriers and excipients. The pharmaceutical composition is effective in attenuating cocaine seeking behavior in a subject when administration into a selected region of a brain of the subject is performed.

According to yet another aspect of the present invention there is provided an article of manufacture which includes: (a) a pharmaceutical composition which includes as an active ingredient a physiologically effective amount of GDNF and physiologically acceptable carriers and excipients; (b) packaging material; and (c) instructions for administration into a selected region of a brain of a subject the pharmaceutical composition as a means of attenuating cocaine seeking behavior in the subject.

According to further features in preferred embodiments of the invention described below, the selected region of a brain includes a nucleus accumbens (NAc)/striatal border.

According to still further features in the described preferred embodiments the physiologically effective amount is in the range of 1 μg to 20 μg, more preferably in the range of 1 to 5 μg, most preferably about 2.5 μg. Alternately, more preferably in the range of 12-18 μg, most preferably about 14 to 15 μg.

According to still further features in the described preferred embodiments controlled release mechanism is selected from the group consisting of an implanted population of cells capable of secreting GDNF, a pump capable of releasing GDNF, and a substrate capable of releasing GDNF bound thereto.

According to still further features in the described preferred embodiments the administration into the selected region of the brain of the subject includes use of a controlled release mechanism.

According to still further features in the described preferred embodiments the controlled release mechanism is identified in the instructions as a means of the administration into the selected region of the brain of the subject the physiologically effective amount of GDNF.

The present invention successfully addresses the shortcomings of the presently known configurations by providing methods of attenuating cocaine-seeking behavior employing glial cell-derived neurotrophic factor (GDNF) and pharmaceutical compositions and articles of manufacture suited for use in practice of the methods.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph illustrating relative transcription of the glial-derived neurotrophic factor (GDNF) gene in SVG cells in response to different substances of abuse. SVG cells were incubated (24 h) with cocaine (n=8), amphetamine (n=3) or morphine (n=3). The resulting mRNA was amplified by reverse transcription-polymerase chain reaction (RT-PCR) performed with GDNF and β-actin primers. Quantitation of RT-PCR products was performed by a densitometric analysis on ethidium bromide images of the gels. The average mean (±SEM) is presented. *P<0.001 vs. control.

FIG. 2. is a bar graph illustrating relative transcription of the D₁ dopamine receptor gene in SVG cells in response to different substances of abuse. SVG cells were incubated (24 hr) with cocaine (n=8), amphetamine (n=3) or morphine (n=3). The resulting mRNA was amplified using reverse transcription-polymerase chain reaction (RT-PCR) performed with D₁ dopamine receptor and β-actin primers. Quantitation of RT-PCR products was performed by a densitometric analysis on ethidium bromide images of the gels. The average mean (±SEM) is presented. *P<0.05 vs. control.

FIG. 3 is a bar graph illustrating the effect of cocaine on transcription of endogenous brain GDNF mRNA. Rats were trained to self-administer cocaine for 11 consecutive days under the FR-1 schedule as detailed hereinbelow. The rat's brains were removed and analyzed for GDNF mRNA expression as in FIG. 1. Means ±SEM (T test, *p<0.05 cocaine versus saline) from six rats are depicted for each panel.

FIGS. 4a and 4b are histograms illustrating cocaine-seeking behavior as a function of time for rats grafted with GDNF-engineered cells. Rats were injected with GDNF— secreting human astrocyte cell line (SVG cells; FIG. 4b this cell line is immortalized but not malignant) or PBS intra-brain (FIG. 4a). Rats subsequently self-administered cocaine for 12 consecutive days under the FR-1 schedule. The rats were exposed to the levers in the operant chambers, and could self-administer cocaine or saline during 1 hr. The mean (±SEM) number of infusions and active lever presses throughout the study is presented. Two way ANOVA with repeated measurements was performed (p<0.001). Means ±SEM from six rats are depicted for each panel.

FIGS. 5a and 5b are histograms illustrating the effect of intra-brain GDNF infusion on cocaine-seeking behavior in rats. Rats received either intra-brain microinjection of PBS (FIG. 5a) or GDNF (FIG. 5b) via a mini-pump and were allowed to self-administer cocaine as in FIGS. 3 and 4.

FIG. 6 is a comparative histogram illustrating the effect of SVG cell transplantation on the behavioral response of rats to available cocaine. Non-treated control (n=10), PBS-injected control (n=4-5) and SVG-implanted (n=6) rats were allowed to self-administer cocaine under the FR-1 schedule. The mean numbers of active lever responses ±SEM are presented. Rats receiving an SVG cell graft displayed lower active lever responses compared to PBS-injected and untreated controls (p<0.0001, main effect of treatment).

FIGS. 7a, 7b, 7c and 7d are micrographs illustrating SV-40 immunohistochemical detection of SVG cells at the site of graft transplantation. Representative sections of SVG cell grafts on the second (A, B) and twelfth (C, D) day following transplantation are presented at 100× (A, C) and 400× (B, D) magnification. The black boxes on the 100× magnification panels demarcate the area that is represented on the adjacent 400× panel. Clusters of SV40-labeled cells in the transplantation tract from the striatum to the nucleus accumbens are stained darkly. Arrows indicate SVG-positive cells. Scale bar=250 μm.

FIG. 8 is a comparative histogram illustrating the effect of GDNF infused into the brain via a mini-pump on cocaine seeking behavior. Non-treated control (n=10) and rats that received a PBS (n=5-7) or GDNF (n=3-7) pump were allowed to self-administer cocaine for 1 hr/day for 12 under the FR-1 schedule. The mean numbers of active lever responses ±SEM are presented. Rats implanted with a GDNF pump have significantly lower numbers of active lever responses compared to PBS pump and untreated controls (p<0.001, main effect of treatment).

FIG. 9 is a comparative histogram illustrating Effect of GDNF-conjugated nanoparticles on the behavioral response to cocaine. Untreated controls (n=10) and rats treated with GDNF-conjugated nanoparticles (n=11), free nanoparticles (n=9), and free GDNF injection (n=5) were allowed to self-administer cocaine as described in FIGS. 4a and 4b. The mean numbers of active lever responses ±SEM are presented as a function of time. Rats receiving an injection of GDNF conjugated nanoparticles displayed lower active lever responses compared to free GDNF injected or free nanoparticle injected or untreated control rats (p<0.0001, main effect of treatment).

FIG. 10 is amicrograph illustrating nanoparticle detection at the site of injection 14 days post treatment. Hemotoxilin histochemical stained tissue reveals clusters of brown-colored nanoparticles from the striatum to the nucleus accumbens (400×).

FIG. 11 is a comparative histogram illustrating the effect GDNF-conjugated nanoparticle treatment on water seeking behavior. Non-treated controls (n—4) and rats that received GDNF-conjugated nanoparticle (n=4) or free nanoparticle injection (n=4) were allowed to self-administer water as described above for cocaine. The mean numbers of active lever responses ±SEM are presented. Treated rats displayed similar active lever responses compared to untreated controls.

FIG. 12 is a comparative histogram illustrating the effect of GDNF-conjugated nanoparticle treatment on cocaine dose-response. Rats learned to self-administer cocaine (1 mg/kg/0.13 ml infusion) for 1 hr/day as described hereinabove. After rats achieved four days of stable (<20% deviation from the mean) levels of bar-pressing for cocaine reinforcement (maintenance), subjects were placed in the self-administration chamber as usual and allowed to self-administer one of three doses of cocaine (0.50, 0.75 or 1.0 mg/kg/infusion). The mean±SEM number of active lever responses is presented. Rats treated with GDNF-conjugated nanoparticles displayed low active lever responses as compared to controls (p<, main effect of treatment).

### DESCRIPTION OF THE PREFERRED EMBODIMENTS

The present invention is of methods of attenuating cocaine-seeking behavior and is further of pharmaceutical compositions and articles of manufacture suited for use in those methods.

Specifically, the present invention employs glial cell-derived neurotrophic factor (GDNF) to attenuate cocaine-seeking behavior. The observed effect, while undeniably physically based, appears, for the first time, to alter the psychological perception of the habituated subject towards cocaine. As a result, it is anticipated that recidivism among individuals that “kick” the cocaine habit according to methods of the present invention will be lower than that typically associated with anti-addiction intervention by previously available alternatives.

The principles and operation of methods, pharmaceutical compositions and articles of manufacture according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Referring now to the drawings, FIGS., 1, 2, 3, 4a and 4b illustrate the inverse relationship between GDNF transcription level and desire for cocaine. Thus, the present invention primarily embodied by a method of attenuating cocaine-seeking behavior in a subject. The method includes administering into a selected region of a brain of the subject a physiologically effective amount of glial cell-derived neurotrophic factor (GDNF) by means of a controlled release mechanism.

The present invention is further embodied by a pharmaceutical composition which includes, as an active ingredient, a physiologically effective amount of GDNF and may further include physiologically acceptable carriers and excipients. The pharmaceutical composition is effective in attenuating cocaine seeking behavior in a subject when administration into a selected region of a brain of the subject is performed. It should be noted that although only direct administration into the brain has been attempted to date, it is envisioned that systemic or peripheral administration of GDNF, with subsequent arrival of GDNF at the desired location in the brain will be achieved during the life of this patent. As such, all such delivery routes are incorporated a priori into the scope of the appended claims. Similarly, positive regulation of endogenous GDNF transcription, is within the the scope of “administering into a selected region of a brain of the subject a physiologically effective amount of glial cell-derived neurotrophic factor” as instantly claimed. Thus, most preferably, administration of the pharmaceutical composition into the selected region of the brain of the subject includes use of a controlled release mechanism. The longer the action of this release mechanism, the more significant the observed effect.

Optionally, but preferably, the pharmaceutical composition is supplied as an article of manufacture which further includes packaging material and instructions for administering the pharmaceutical composition into a selected region of a brain of a subject as a means of attenuating cocaine seeking behavior in the subject. In an article of manufacture, the controlled release mechanism is preferably identified in the instructions as a means of administering the physiologically effective amount of GDNF into the selected region of the brain of the subject.

It is currently believed that a region of the brain which includes the NAc/striatal border is optimum for GDNF activity, although application of GDNF to other sites in the brain is well within the scope of the claimed invention.

According to some preferred embodiments the physiologically effective amount is in the range of 1 μg to 20 μg of GDNF/day per subject. In rats the effective dose is preferably in the range of 1 μg to 5 μg, most preferably about 2.5 μg as detailed hereinbelow. Previous work in Parkinson's disease suggests that in humans the effective dose is in the range of 12-18 μg, most preferably about 14 to 15 μg (see, for example, Gill et all; 2003).

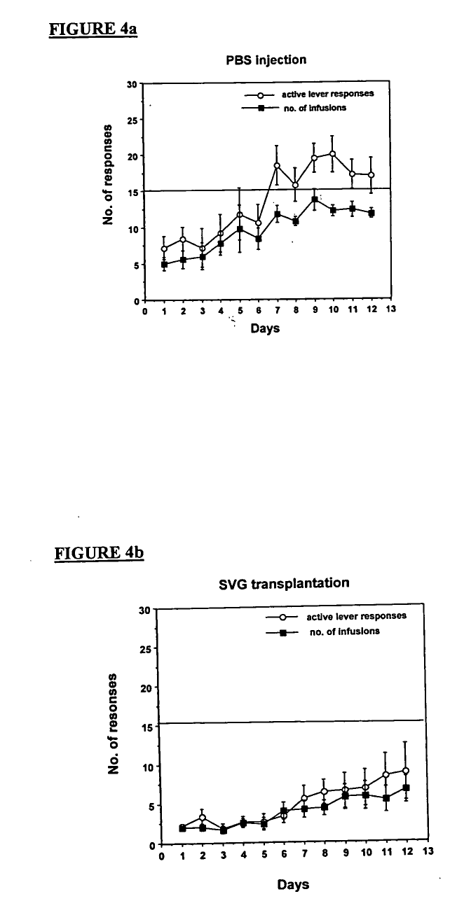

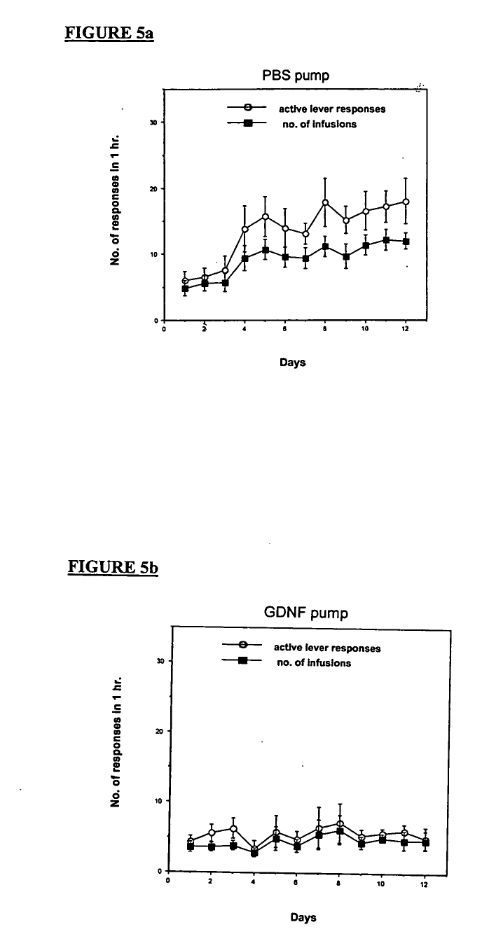

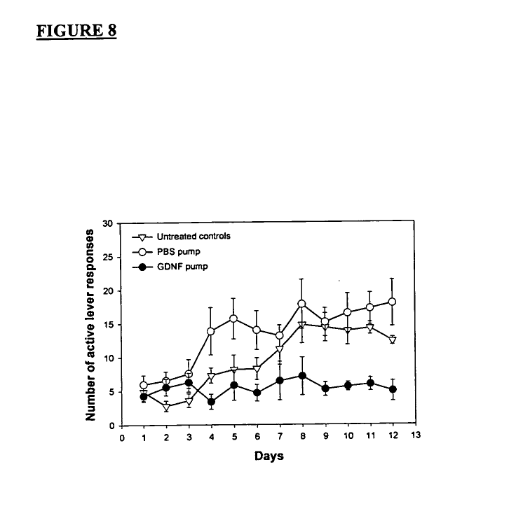

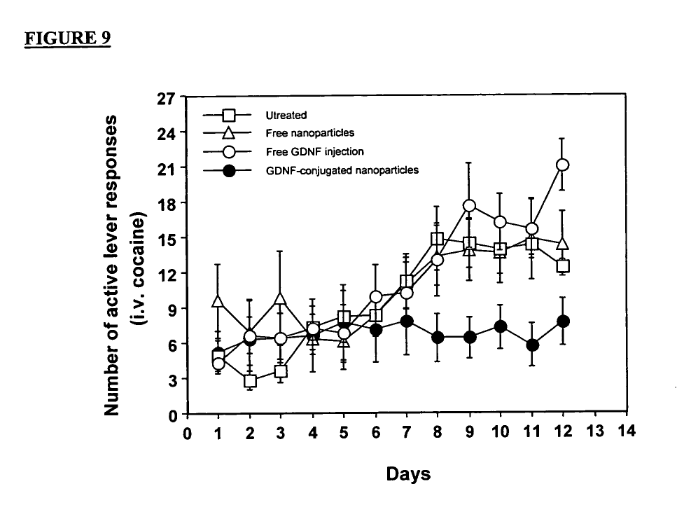

Controlled release mechanism, as used in this specification and the accompanying claims include, but are not limited to, an implanted population of cells capable of secreting GDNF (e.g. SVG cells as described hereinbelow; see FIGS. 4b, 6, 7a-d, a pump capable of releasing GDNF (see FIGS. 5b and 8), and a substrate capable of releasing GDNF bound thereto (see FIGS. 9-12).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al.; (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes 1-111 Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Before presenting examples, reference is made to the following materials and methods employed in performance of experiments described in the examples.

Materials and Methods:

Animals: Male Sprague Dawley rats weighing 230-280 g (Bar-Ilan University) were maintained on a 12 h-12 h light dark cycle with free access to food and water.

SVG Cell Culture: Human SVG astrocytes (Major et al., 1985) were grown under sterile conditions in supplemented Eagle's minimum essential medium (E-MEM; Biological Industries Ltd., Beit Haemek, Israel) supplemented with 10% fetal calf serum, 5 mM glutamine and 50 μg/ml gentamycin (Biological Industries). The cells were grown as a monolayer on untreated plasticware at 37° C. and 5% CO₂. The medium was routinely changed every 4 days, and the cells were passaged near confluence every 8 days.

Surgeries: Rats were maintained on chloral hydrate (400 mg/kg, intraperitoneally, Merck, Darmstadt, Germany) throughout the surgical procedures. All experimental procedures were approved by the University Animal Care and Use Committees and were done in accordance with National Institutes of Health guidelines.

SVG cell transplantation: SVG cell transplantation was conducted as previously described (Tomatore et al., 1996). After SVG cells were removed from the dish with 0.025% trypsin, the cell suspension was diluted in PBS to the concentration of 1×10⁶ live cells/8 μl (Tornatore et al., 1996). The cells were grafted into a 4 mm tract in a volume of 0.5 1 every 0.25 mm according to the following stereotaxic coordinates measured from Bregma: A: 1.6, L: 1.6, V: −8 to −4 mm. The cell suspension (an 8 μl solution of 10⁶ cells) was injected in a volume of 0.5 mevery 0.25 mm over the 4 mm tract. PBS-injected controls received 8 1 of PBS to the same stereotaxic coordinates, while untreated controls did not receive stereotaxic surgery.

Mini-pump implantation: Some animals received intra-brain infusion of GDNF or PBS by subcutaneous implantation of an osmotic mini-pump (Alzet Model 1002, Alza Corp. Palo Alto, Calif.) and some rats were left as untreated controls. Pumps were filled with GDNF (PeproTech Asia CytoLab Ltd., Rehovot, Israel) diluted in PBS at 0.41 microgram/microliter. Minipumps were calibrated to deliver 2.5 μg/day for 14 days. The GDNF concentration in the minipump was chosen, because it is on the low end of doses of minipump-infused GDNF that are effective at blocking morphine-induced increases in VTA tyrosine hydroxylase immunoreactivity (Messer et al., 2000) and preventing the degeneration of substantia nigra dopaminergic neurons after a neurotoxic lesion (Lu & Hagg, 1997). Further, this dose was effective at blocking the rewarding effects of cocaine in the conditioned place preference paradigm (Messer et al., 2000). Finally, it is within the range of doses that were found to be effective in humans (Gill et al., 2003). The pump cannula was implanted into the NAc/striatal border using the following stereotaxic coordinates measured from Bregma: A: 1.6 mm, L: 1.6 mm, V: −6.5.

Nanoparticle injection: GDNF (Cytolab Ltd. (Peprotech Asia), Rehovot, Israel) was conjugated to at a concentration of 0.22 μg GDNF/0.015 mg nanoparticles/μl. Nanoparticle, with or without GDNF, were injected in a volume of 0.5 μl every 0.25 mm into a 4 mm tract (NAc core to striatum) according to the following stereotaxic coordinates measured from Bregma: A: 1.6, L: 1.6, V: −8 to −4 mm. In the same rats, free nanoparticles or GDNF-conjugated nanoparticles were also injected into a 1.8 mm tract (NAc shell) according to the following coordinates: A: 1.6, L: 0.8, V: −8 to −6.2. In a third group of rats, 0.41 g/1 of free GDNF in a volume of 8 1 was injected into tracts using the same coordinates that were mentioned above. Finally, the untreated control group did not receive stereotaxic surgery or intra-brain injections.

Intravenous catheterization: Rats that were subjects in the cocaine self-administration experiments were also implanted with intravenous silastic catheters (Dow Corning, Midland, Mich.) into the right jugular vein (Roth-Deri, 2003). The catheter was secured to the vein with silk sutures and was passed subcutaneously to the top of the skull where it exited into a connector (a modified 22 gauge cannula; Plastics One, Roanoke, Va.) mounted to the skull with Mx-80 screws (Small Parts, Inc., Miami Lakes, Fla.) and dental cement (Yates & Bird, Chicago, Ill.).

Cocaine self-administration: Rats were trained to self-administer cocaine as previously described (Roth-Deri, 2003). Briefly, four days after catheterization and treatment, rats were transferred to operant conditioning chambers (Med-Associates, Inc., Georgia, Vt.) for one hour daily for 12 days during their dark cycle and allowed to self-administer intravenous cocaine (1.0 mg/kg per 0.13 ml infusion, 20 sec) (obtained from the National Institutes on Drug Abuse, Research Technology Branch, Rockville, Md.) under a fixed-ratio-I schedule of reinforcement. During the 20-sec cocaine infusion, active lever presses were recorded, but no additional cocaine reinforcement was provided. The acquisition of cocaine self-administration was measured via active lever responses, infusions, and inactive lever responses in untreated controls and in rats treated with implanted SVG cells, minipump implanted, PBS-injected, free nanoparticles, free GDNF, or GDNF-conjugated nanoparticles.

After reaching maintenance levels, rats were again placed in the operant conditioning chamber and allowed to self-administer cocaine. Certain groups of rats (GDNF-conjugated nanoparticle treated, and untreated control were given the same dose of cocaine as the training dose (1 mg/kg/infusion). Different groups of rats were given 0.75 mg/kg/infusion (GDNF-conjugated nanoparticles; untreated control, or 0.50 mg/kg/infusion (GDNF-conjugated nanoparticles; untreated control, for one session only. The number of active lever responses, reinforcements, and inactive lever responses were measured in untreated control and in GDNF-conjugated nanoparticle treated rats.

Water self-administration: A separate group of rats that did not undergo i.v. catheterization were trained to bar press for water reinforcement (Green-Sadan et al. (2003) Eur. J. of Neuroscience 18:2093-2098) 4 days after no treatment (untreated controls) or injection with free GDNF or GDNF-conjugated nanoparticles. Animals were allowed 15 ml of water per day in addition to approximately 13 ml of water consumed during the daily sessions. The operant chambers, reinforcement schedule, and session duration were the same as those used for cocaine self-administration. Rats received 0.13 ml of water per lever press, delivered into a drinking dish (ENV-200R3AM, Med-Associates, Inc.) in the operant chamber. Active lever responses, reinforcements, and inactive lever responses were recorded. The acquisition of water was measured via active lever responses, infusions and inactive lever responses.

Hematoxilin histochemistry: Nanoparticle-injected animals underwent perfusion with 4% paraformaldahyde from the left cardiac ventricle. Their brains were then removed, immersed in paraformaldehyde overnight, in 20% sucrose (Frutarom Meer Corp., North Bergen, N.J.) for 48 hours, and then frozen on dry ice. Thirty micron sections of perfused brains were cut using a cryostat (Leica CM-1800, Chatsworth, Calif.) and then stained using incubation with hemotoxilin for 30 sec. The sections were then dipped in distilled water for 1 sec, and held under running water for 5 min. Afterward the sections were dehydrated using increasing concentrations of ethanol, immersed in 70% alcohol for 3 min, then 70% alcohol with 2N HCL, next 95% alcohol for 5 min and finally immersed consecutively in 100% alcohol, for 10 min. Finally, the sections were successively incubated with xylene for 10 min. Nanoparticles were visualized using a light microscope (AH3-RFCA, Olympus Microscopy, Hamburgh, Germany) at 100× and 400× and photographed using a digital camera (DP-50, Olympus).

SV40 immunohistochemistry: SV40 immunohistochemistry was performed as described previously (Tornatore et al., 1996). Fifteen micron sections of frozen perfused brains 24 hours following cocaine self-administration were cut using a cryostat (Leica CM-1800, Chatsworth, Calif.). Free-floating sections were incubated for 1 hour in a PBS blocking solution containing 1% BSA and 0.3% Triton X-100 and subsequently incubated with a monoclonal antibody, mouse anti SV-40 T-antigen, 1:200 (Chemicon International, Temecula, Calif.) overnight at 4° C. The next day, the sections were washed and incubated for 1 hour at 37° C. with alkaline phosphatase-conjugated goat anti mouse IgG 1:500 (Chemicon). Sections were then washed and developed using the BCIP/NBT liquid substrate system (Sigma-Aldrich, St. Louis, Mo.). Secondary antibody in the absence of the primary antibody was used as a negative control. Cells were visualized using a light microscope (AH3-RFCA, Olympus Microscopy, Hamburgh, Germany) at 100× and 400× and photographed using a digital camera (DP-50, Olympus).

Reverse Transcriptase-Polymerase Chain Reaction (RT-PCR)

Twenty-four hours after the last cocaine self-administration trial, the animals were anesthetized with chloral hydrate and decapitated. Tissue punches from the striatum and nucleus accumbens were rapidly taken as described (Zangen et al., 1999). The tissue samples were immediately frozen on dry ice and stored at −70° C. until RNA extraction.

Total RNA was isolated from rat brain tissue by the single-step method using the commercially available TriReagent™ (Sigma, Rehovot, Israel) (Kinor et al., 2001).

First-strand cDNA synthesis was carried out in a final reaction volume of 20 μl (Kinor et al., 2001). RT-PCR was carried out on the resulting cDNA in a final reaction volume of 50 μl. First-strand cDNA (2 μl) was added to the PCR mixture containing: 0.2 mM dNTP mix, 1 mM of each oligonucleotide primer and 2.5 U Taq DNA polymerase (Roche, Mannheim, Germany) in the buffer supplied by the manufacturer (Roche). Primers sequences described in Green-Sadan et al. ((2003) Eur. J. of Neuroscience 18:2093-2098) are fully incorporated herein by reference.

Reactions were initially denatured at 94° C. for 2 min. PCR was then performed using a thermal cycler (MJ Research, Watertown, Mass.) programmed for 35 cycles. Each cycle was: 1 min at 94° C., 1 min at 55° C., and 1 min at 72° C. Optimal conditions for the detection of the GDNF (35 cycles) and eta-actin 29 cycles) were determined. The PCR products were analyzed on 1% agarose els containing ethidium bromide. Image densitometric analysis was performed using the NIH Image software developed by David Chow, Division of Computer Research and Technology, NIH, 1998 edition.

Statistical Analysis: In the SVG cell and minipump experiments, a Student t-test was used to determine differences in GDNF mRNA between saline- and cocaine-exposed groups. A one-way ANOVA (treatment) with repeated measures (days) was employed to examine the effect of the experimental treatments on active lever presses for each experiment followed by a post-hoc one-way ANOVA to determine which treatment groups were altered. Data are presented as means ±SEM. Groups were considered significantly different if p<0.05.

In the nanoparticle experiments, a one-way ANOVA (treatment) with repeated measures (days) was employed to examine the effect of the experimental treatments on active lever presses for each experiment followed by a Student-Newman-Keuls post-hoc test to determine which treatment groups were altered. A two-way ANOVA (treatment×infusion dose of cocaine) was employed to examine the effect of GDNF-conjugated nanoparticles on cocaine self-administration using several cocaine doses. A one-way ANOVA followed by a Student-Neuman-Keuls post-hoc test was employed to examine the effect of dose on active lever presses in each group. Data are presented as means±SEM. Groups were considered significantly different if p<0.05.

### EXAMPLE 1

### Cocaine Stimulates GDNF Expression in SVG Cells

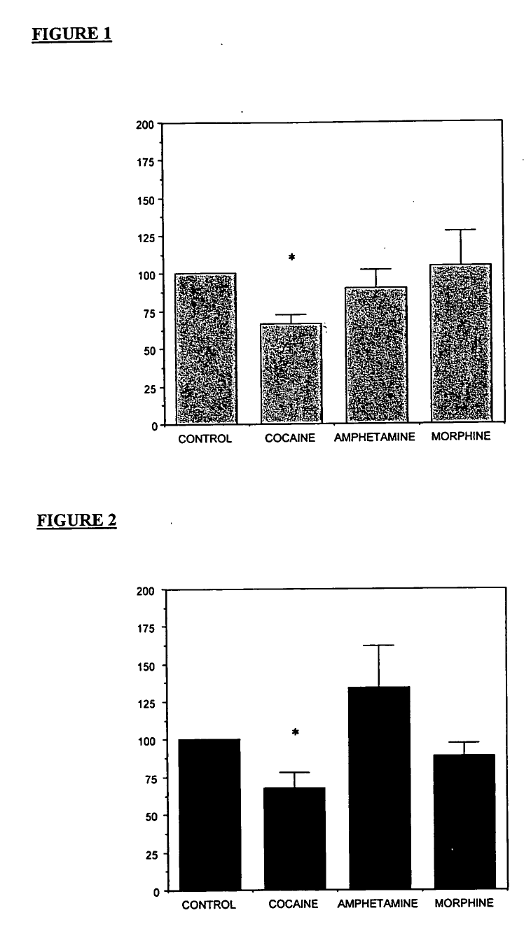

The SVG cell line is a human fetal astrocyte cell line (Major et al., 1985), which provides several glial functions, including the release of GDNF (Yadid et al., 1999). It is known that a functional D₁ dopamine receptor is present in these cells and that activation of this receptor causes an increase in GDNF transcription and production, mediated via intracellular free calcium. In order to demonstrate that cocaine has a specific effect on GDNF transcription, SVG cells were incubated in the presence of cocaine, morphine or amphetamine. A twenty-four hour incubation of SVG cells with cocaine significantly lowered D₁ receptor (FIG. 2) and GDNF (FIG. 1) mRNA levels. Amphetamine and morphine had no significant effect. These results indicate that cocaine has a direct and specific effect on extraneuronal cells, in addition to its known effect on the neuronal dopamine transporter. The observed decrease in GDNF neurotrophic support may increase local neuronal vulnerability.

### EXAMPLE 2

### Effect of Cocaine on GDNF Expression in Rat Brain

In order to determine the effects of cocaine in vivo, rats were permitted to self-administer cocaine. In cocaine self-administration trials, for 14 consecutive days, rats received 1-hr daily training sessions during their dark cycles (rats were maintained in a 12-hr light-12-hr dark cycle). Each operant box had two levers located 9 cm above the floor of the chamber. When the “active” lever was pressed the infusion pump, which caused an i.v. infusion of cocaine (1 mg/kg/0.13 ml during 20 sec) was activated and the number of presses was recorded. When the “inactive” lever was pressed, the number of presses was recorded, but the infusion pump was not activated. During drug administration, a white light, located above the operating lever lit up for 20 sec. Bar presses during these 20 sec were counted, but did not cause further infusions of cocaine. Thus, the self-administration of cocaine was under a fixed-ratio-1 (FR-1; each lever press caused one cocaine injection) schedule of reinforcement (Shaham (1995) psychopharmacology 119(3): 334-341).

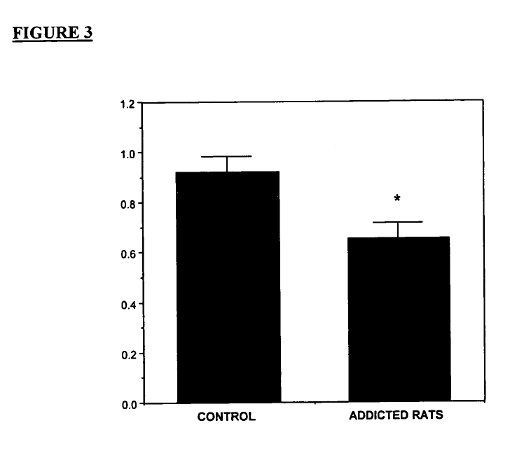

Analysis of the brain of rats permitted to consume cocaine showed a marked decrease in GDNF mRNA (FIG. 3) relative to control rats. GDNF mRNA levels were evaluated in tissue punches taken from the striatum of rats chronically exposed to cocaine using the self-administration technique. Rats self-administered 9.6±0.9 mg/kg of cocaine per day for 12 days. Animals that self-administered cocaine had a 69% reduction (p<0.001) in striatal levels of GDNF mRNA (GDNF/eta-actin mRNA: non cocaine-treated controls: 0.97+0.07, cocaine-treated: 0.30±0.05), while no difference was detected in nucleus accumbens GDNF mRNA levels (GDNF/eta-actin mRNA: controls: 0.4+0.08 cocaine: 0.4±0.03).

These data support the idea that GDNF is relevant for neuronal protection and decrease during cocaine exposure causing neuronal vulnerability to increase and are in accord with the data from Example 1.

### EXAMPLE 3

### Correlation Between Decreased GDNF Expression and Cocaine Seeking Behavior

Using the self-administration paradigm described in example 2, the effect of GDNF administration into the brain on cocaine-seeking behavior was examined. Administration of GDNF was by intra-brain injection of SVG cells as described hereinabove. In rats treated with PBS (negative controls), the active lever was routinely pressed even while the last dose of cocaine was still being administered (FIGS. 4a and 5a). This is indicative of a desire to increase the drug dose and correlates to drug seeking behavior in human cocaine users. In sharp contrast rats treated with GDNF released from implanted SVG cells (FIG. 4b) or a pump delivering GDNF (FIG. 5b) rarely pressed the active lever while the previous dose of drug was being administered. In addition, these rats requested far fewer cocaine infusions than their PBS treated counterparts.

### EXAMPLE 4

### Correlation Between Decreased GDNF Expression and Cocaine Seeking Behavior Supplementary Data

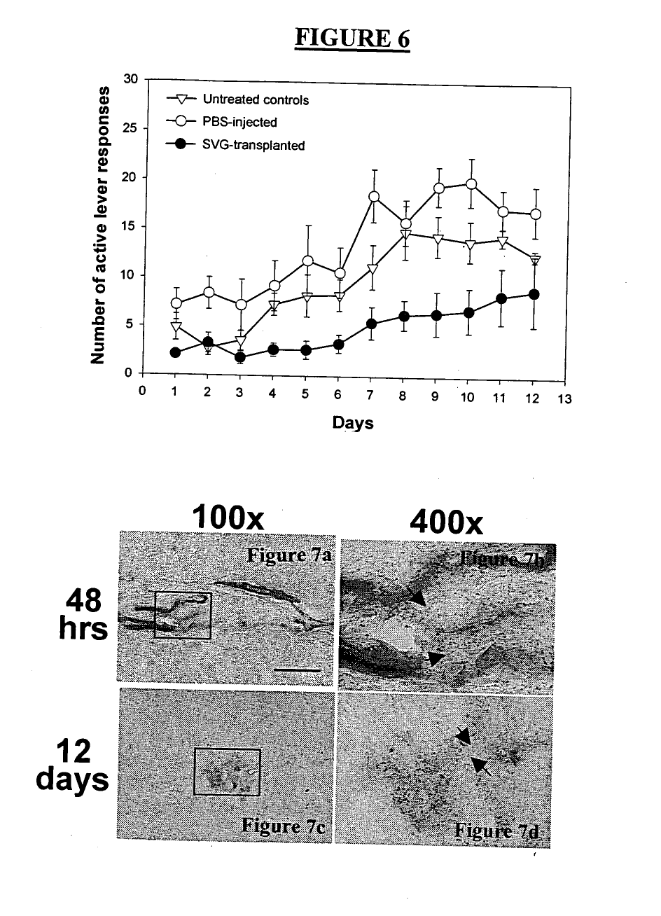

In an additional experiment it was determined that there is a main effect of SVG-cell transplantation treatment [F (2,187)=12.893; p<0.001] and a main effect of days [F (11,187)=14.164; p<0.0001] on the number of active lever presses. Post-hoc tests revealed that in control rats that were either not treated or received a PBS injection, there is a steady increase in active lever presses in response to cocaine [untreated control: F (11,108)=6.787; p<0.0001; PBS injection: F (11, 104)=8.421; p<0.0001] (FIG. 6,). Rats that received a tract of SVG cells transplanted into the striatum and NAc show an attenuated behavioral response to cocaine compared to PBS-injected (p<0.0001) and untreated control (p<0.0001) rats. This supports the hypothesis that GDNF reduces cocaine-seeking behavior.

The number of infusions showed a trend similar to the number of active lever presses (data not shown). Inactive lever responses were consistently low (untreated controls: 2.79±0.23, PBS-injected: 6.20±0.57, SVG-implanted: 5.25+0.55) and were significantly different than active lever responses in PBS-injected (p<0.0001) and untreated (p<0.0001) control groups.

In a separate experiment, it was determined that rats that received SVG cell transplants did not demonstrate disrupted operant behavior maintained by water reinforcement. There was no main effect of treatment, although there was a main effect of days (F (11, 154)=6.32; p<0.001). Therefore, SVG-implanted rats did not show significant differences in active lever responses for water reinforcement after water deprivation compared to PBS-injected and untreated control rats. The mean lever presses for each group for the 12 experimental days were: untreated control: 75.10±3.23, PBS-injected: 84.0±3.31, SVG-implanted: 76.5±3.02, while the mean inactive lever presses were: untreated control: 13.2±1.05, PBS-injected: 14.8±1.89, SVG-implanted: 13.3±0.92.

### EXAMPLE 5

### Persistence Of Transplanted SVG Cells

In order to determine the persistence of transplanted cells, a histochemical analysis of brain sections was performed as described hereinabove. SVG immunohistochemistry revealed clusters of SV40-labeled cells in the transplantation tract two days post-transplantation (FIGS. 7a and 7b), some SV40-labeled cells in the healed tract twelve days after transplantation (FIGS. 7c and 7d), and virtually no labeling eighteen days after transplantation (not shown). This indicates that the observed increase in cocaine consumption in SVG transplanted animals during the 12-day study may be a result of decreased GDNF secretion, as opposed to a decrease in the effectiveness of GDNF. Decreased GDNF is apparently due to a gradual decrease in SV40 labeling which indicates less SVG cells and is, in turn, correlated to the increase in response to cocaine

### EXAMPLE 6

### Administration of GDNF via a Minipump Reduces Cocaine Seeking Behavior

In order to confirm that the initial decrease in cocaine seeking behavior in SVG implanted rats was directly attributable to GDNF, and to confirm that the observed increase in cocaine consumption in SVG transplanted animals during the 12 day study resulted from decreased GDNF delivery (as opposed to decreased effect of delivered GDNF) mini-pumps loaded with GDNF were implanted as described hereinabove.

A significant effect of treatment [F (2, 143)=9.574; p<0.01], days [F (11, 143)=6.089; p<0.0001], and an interaction between treatment and days [F (22, 143)=1.848; p<0.05] on active lever presses were observed (summarized graphically in FIG. 8). Post-hoc tests reveal that control rats that were either untreated or received a chronic PBS infusion into the NAc showed a gradual increase in active lever responses during the course of the experiment [untreated controls: F (11,108)=6.787; p<0.0001; PBS pump: F (11, 41)=2.219; p<0.05] (FIG. 8). However, rats that received a chronic infusion of GDNF exhibited a weak response to cocaine (FIG. 8). Further, when the three treatment groups were compared, each group was significantly different than the others in active lever presses (p<0.0001).

The number of infusions showed a trend similar to the number of active lever presses (data not shown). Inactive lever responses were consistently low (untreated controls: 2.79+0.23, PBS pump: 5.28±0.49, GDNF pump: 3.32±0.30) and significantly different than active lever responses in the PBS pump (p<0.0001) and untreated (p<0.0001) control groups.

These data confirm the efficacy of GDNF in reducing cocaine-seeking behavior and suggest that increased cocaine consumption among SVG cell transplanted rats resulted from decreased cell numbers, and not from decreased efficacy of GDNF.

### EXAMPLE 7

### Presence Of GDNF-Associated Nanoparticles in the Brain Attenuates Cocaine Seeking Behavior

Since it was established that GDNF-loaded minipumps which delivered 2.5 g/day were effective at attenuating cocaine seeking behavior, and since conjugation of GDNF to nanoparticles decreases efficacy by roughly fifty percent nanoparticle solutions with or without 0.2 μg GDNF/0.16 mg nanoparticles/μl were injected in a volume of 0.5 μl every 0.25 mm into a 4 mm tract as described hereinabove. Animals were allowed to self-administer cocaine as described hereinabove.

There is a main effect of treatment {F (3,124)=6.08; p<0.01] (<<repeated measures) on the number of active lever presses during maintenance (days 8-12). A Student-Newman-Keuls post-hoc test demonstrated that rats that received GDNF-conjugated nanoparticles injected into a tract in the striatum and NAc show a weak behavioral response to cocaine compared to untreated control (p<0.05), free nanoparticle-injected (p<0.05), and free GDNF-injected (p<0.05) rats (FIG. 9).

The number of infusions showed a trend similar to the number of active lever presses (data not shown). Inactive lever responses were consistently low (untreated controls: 2.79±0.23, free nanoparticles: 3.33±0.39, free GDNF: 4.54+0.35, GDNF-conjugated nanoparticles: 4.77±0.23) and were significantly different from active lever responses on days 8-12 in untreated control {F (1, 72)=89.657; p<0.0001], free nanoparticle {F (1, 48)=14.024; p<0.01], and free GDNF {F (1, 28)=38.086; p<0.001] groups, but not in the GDNF-conjugated nanoparticle group.

These data indicate that GDNF-conjugated nanoparticles represent an effective delivery vehicle to the brain and are useful in attenuating drug-seeking behavior over the course of time.

### EXAMPLE 8

### Persistence of Nanoparticles as Analyzed by Histochemistry

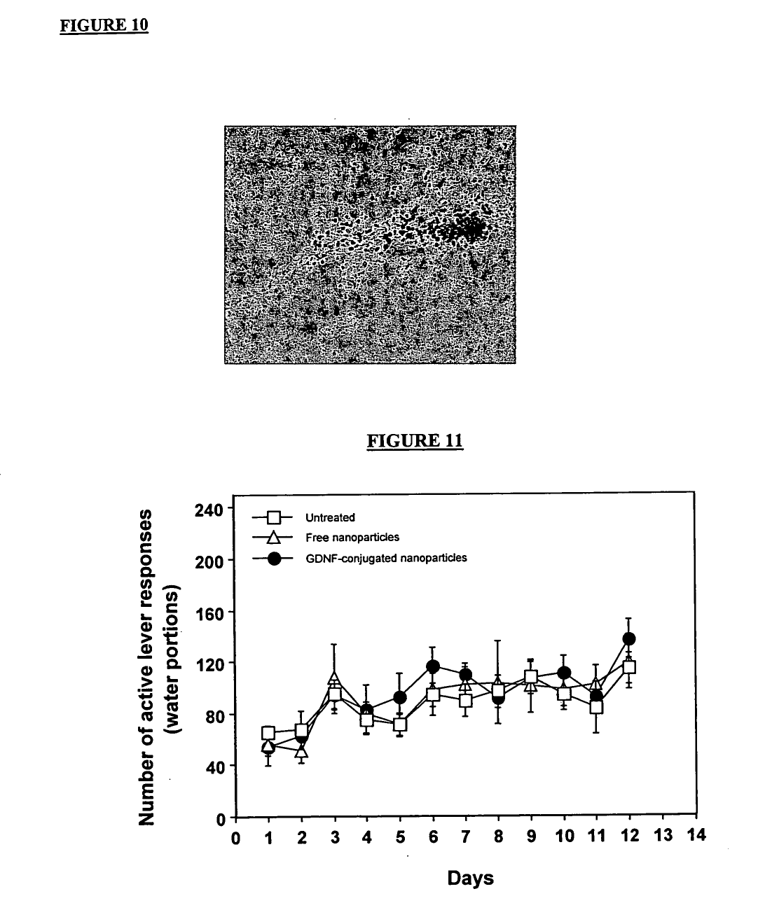

Hemotoxilin histochemistry (described hereinabove) revealed that nanoparticles were clustered in the transplantation tract at fourteen days post-transplantation (FIG. 10). This result indicates that the particles are not subject to unwanted dispersion.

### EXAMPLE 9

### GDNF Effect is Specific for Cocaine Seeking Behavior

In a separate experiment, rats that received GDNF-conjugated nanoparticles were permitted to demand water in a system similar to that used for cocaine administration (see materials and methods hereinabove). These rats did not demonstrate disrupted operant behavior maintained by water reinforcement.

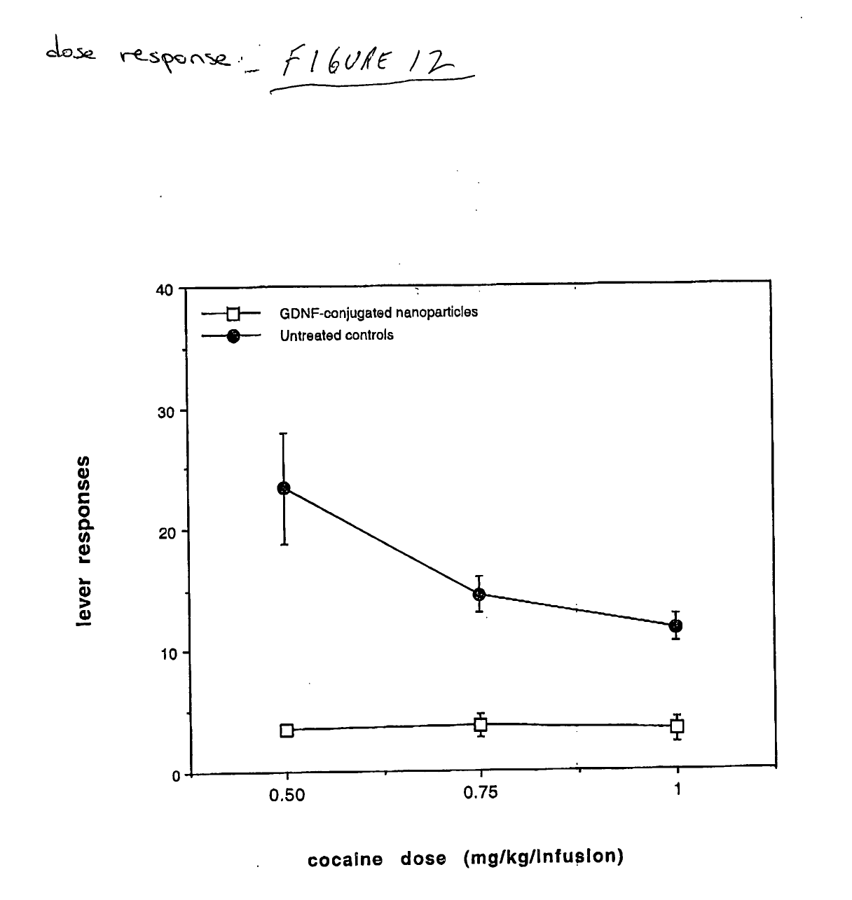

There was no main effect of treatment, although there was a main effect of days {F (4,36)=4.317; p<0.01] (FIG. 11). Thus, rats that were microinjected with GDNF-conjugated nanoparticles did not show significant differences in active lever responses for water reinforcement after water deprivation compared to untreated control and free nanoparticle-injected rats. The mean inactive lever presses were low (untreated control: 10.70±2.41, free nanoparticles: 12.06±1.91, GDNF-conjugated nanoparticles: 7.86±1.42) and were significantly different than active lever responses on days 8-12 in all groups (untreated control {F (1, 24)=26.562; p<0.01], free nanoparticles {F (1, 24)=16.718; p<0.01], and GDNF-conjugated nanoparticles {F (1, 24)=252.209; p<0.0001]).

This result indicates that the observed effect of GDNF in example 7 does not result from a general behavioral change (e.g. lethargy or confusion) but is indicative of a decrease in the level of desire for cocaine.

### EXAMPLE 10

### GDNF—Conjugated Nanoparticles Influencecocaine Dose-Response

In an additional separate experiment, rats that received GDNF-conjugated nanoparticles were permitted to train themselves to habitually use cocaine until they reached maintenance levels. These rats were then divided into dosage groups and continued in the operant conditioning chamber and allowed to self-administer cocaine.

Some rats (GDNF-conjugated nanoparticle treated, and untreated control) were allowed to self administer the same dose of cocaine as the training dose (1 mg/kg/infusion).

Additional rats were allowed to self administer 0.75 mg/kg/infusion (GDNF-conjugated nanoparticles; untreated control).

Another additional set of rats were allowed to self administer 0.50 mg/kg/infusion (GDNF-conjugated nanoparticles; untreated control, for one session only). The number of active lever responses, reinforcements, and inactive lever responses were measured in untreated control and in GDNF-conjugated nanoparticle treated rats. Results are summarized graphically in FIG. 12.

There was a significant main effect of treatment [F(1,31)=43.09; p<0.0001] on the number of active lever responses for cocaine. Rats treated with GDNF-conjugated nanoparticles showed a lower number of active lever presses at all three doses (0.50, 0.75 and 1 mg/kg/infusion) compared to untreated controls (FIG. 12). Further, for rats that did not receive GDNf there was a significant effect of dose [F(2,23)=5.267; p<0.05] on the number of active lever responses for cocaine (i.e. lower dose was compensated by additional lever presses). In summary, control rats pressed more on the active lever for the 0.50 mg/kg/infusion (p<0.05) dose than for the 0.75 and 1 mg/kg/infusion dose (FIG. 12), while rats that received GDNF-conjugated nanoparticles did not do so.

These results indicate that GDNF not only attenuates cocaine-seeking behavior, it makes a habituated user more amenable to a reduction in dose. Specifically, rats habituated to a specific dose did not attempt to compensate for a reduced dose by additional lever presses.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

Airaksinen, M. S., Titievsky, A. & Saarma, M. (1999) GDNF family neurotrophic factor signaling: four masters, one servant? Mol. Cell. Neurosci., 13, 313-325.
Beck K D, Valverde J, Alexi T, Poulsen K, Moffat B, Vandlen R A, Rosenthal A, Hefti F. Mesencephalic dopaminergic neurons protected by GDNF from axotomy-induced degeneration in the adult brain. Nature. 1995 Jan. 26;373(6512):339-41.
Beitner-Johnson, D., Guitart, X. & Nestler, E. J. (1992) Neurofilament proteins and the mesolimbic dopamine system: common regulation by chronic morphine and chronic cocaine in the rat ventral tegmental area. J. Neurosci., 12, 2165-2176.
Berhow, M. T., Russell, D. S., Terwilliger, R. Z., Beitner-Johnson, D., Self, D. W., Lindsay, R. M. & Nestler, E. J. (1995) Influence of neurotrophic factors on morphine- and cocaine-induced biochemical changes in the mesolimbic dopamine system. Neuroscience, 68, 969-979.
Blakemore, W. F. & Franklin, R. J. (1991) Transplantation of glial cells into the CNS. Trends Neurosci., 14, 323-327.
Boundy, V. A., Gold, S. J., Messer, C. J., Chen, J., Son, J. H., Joh, T. H. & Nestler, E. J. (1998) Regulation of tyrosine hydroxylase promoter activity by chronic morphine in TH9.0-LacZ transgenic mice. J Neurosci, 18, 9989-9995.
Bowman, R. E., Beck, K. D. & Luine, V. N. (2003) Chronic stress effects on memory: sex differences in performance and monoaminergic activity. Horm. Behav., 43, 48-59.
Carroll, F. I., Howell, L. L. & Kuhar, M. J. (1999) Pharmacotherapies for treatment of cocaine abuse: preclinical aspects. J. Med. Chem., 42, 2721-2736.
Costantini, L. C. & Isacson, 0. (2000) Immunophilin ligands and GDNF enhance neurite branching or elongation from developing dopamine neurons in culture. Exp. Neurol., 164, 60-70.
Gash, D. M., Zhang, Z., Ovadia, A., Cass, W. A., Yi, A., Simmerman, L., Russell, D., Martin, D., Lapchak, P. A., Collins, F., Hoffer, B. J. & Gerhardt, G. A. (1996) Functional recovery in parkinsonian monkeys treated with GDNF. Nature, 380, 252-255.
Georgievska, B., Kirik, D. & Bjorklund, A. (2002) Aberrant sprouting and downregulation of tyro sine hydroxylase in lesioned nigrostriatal dopamine neurons induced by long-lasting overexpression of glial cell line derived neurotrophic factor in the striatum by lentiviral gene transfer. Exp. Neurol., 177, 461-474.
Gill, S. S., Patel, N. K., Hotton, G. R., O'Sullivan, K., McCarter, R., Bunnage, M., Brooks, D. J., Svendsen, C. N. & Heywood, P. (2003) Direct brain infusion of glial cell line-derived neurotrophic factor in Parkinson disease. Nat. Med., 9, 589-595.
Horger, B. A., Iyasere, C. A., Berhow, M. T., Messer, C. J., Nestler, E. J. & Taylor, J. R. (1999) Enhancement of locomotor activity and conditioned reward to cocaine by brain-derived neurotrophic factor. J. Neurosci., 19, 4110-4122.
Kearns, C. M. & Gash, D. M. (1995) GDNF protects nigral dopamine neurons against 6-hydroxydopamine in vivo. Brain Res., 672, 104-111.
Kinor, N., Geffen, R., Golomb, E., Zinman, T. & Yadid, G. (2001) Dopamine increases glial cell line-derived neurotrophic factor in human fetal astrocytes. Glia, 33, 143-150.
Koob, G. F., Sanna, P. P. & Bloom, F. E. (1998) Neuroscience of addiction. Neuron, 21, 467-476.
Lin LF, Doherty DH, Lile JD, Bektesh S, Collins F. GDNF: a glial cell line-derived neurotrophic factor for midbrain dopaminergic neurons. Science. 1993 May 21;260(5111):1130-2.
Lipton, J. W., Ling, Z., Vu, T. Q., Robie, H. C., Mangan, K. P., Weese-Mayer, D. E. & Carvey, P. M. (1999) Prenatal cocaine exposure reduces glial cell line-derived neurotrophic factor (GDNF) in the striatum and the carotid body of the rat: implications for DA neurodevelopment. Brain Res. Dev. Brain Res., 118, 231-235.
Lu, X. & Hagg, T. (1997) Glial cell line-derived neurotrophic factor prevents death, but not reductions in tyrosine hydroxylase, of injured nigrostriatal neurons in adult rats. J. Comp. Neurol., 388, 484-494.
Major, E. O., Miller, A. E., Mourrain, P., Traub, R. G., de Widt, E. & Sever, J. (1985) Establishment of a line of human fetal glial cells that supports JC virus multiplication. Proc. Natl. Acad. Sci. U.S.A., 82, 1257-1261.
Melega, W. P., Lacan, G., Desalles, A. A. & Phelps, M. E. (2000) Long-term methamphetamine-induced decreases of [(11C]WIN 35,428 binding in striatum are reduced by GDNF: PET studies in the vervet monkey. Synapse, 35, 243-249.
Messer, C. J., Eisch, A. J., Carlezon, W. A., Jr., Whisler, K., Shen, L., Wolf, D. H., Westphal, H., Collins, F., Russell, D. S. & Nestler, E. J. (2000) Role for GDNF in biochemical and behavioral adaptations to drugs of abuse. Neuron, 26, 247-257.
Mitchell, J. B. & Meaney, M. J. (1991) Effects of corticosterone on response consolidation and retrieval in the forced swim test. Behav. Neurosci., 105, 798-803.
Nagtegaal, I. D., Lakke, E. A. & Marani, E. (1998) Trophic and tropic factors in the development of the central nervous system. Arch. Physiol. Biochem., 106, 161-202.
Nassogne, M. C., Louahed, J., Evrard, P. & Courtoy, P. J. (1997) Cocaine induces apoptosis in cortical neurons of fetal mice. J. Neurochem., 68, 2442-2450.
Nestler, E. J. & Aghajanian, G. K. (1997) Molecular and cellular basis of addiction. Science, 278, 58-63.
Nestler EJ, Hope BT, Widnell K L. Drug addiction: a model for the molecular basis of neural plasticity. Neuron. 1993 December;11(6):995-1006.
Parish, C. L., Stanic, D., Drago, J., Borrelli, E., Finkelstein, D. I. & Home, M. K. (2002) Effects of long-term treatment with dop amine receptor agonists and antagonists on terminal arbor size. Eur. J. Neurosci., 16, 787-794.
Pierce, R. C. & Bari, A. A. (2001) The role of neurotrophic factors in psychostimulant-induced behavioral and neuronal plasticity. Rev. Neurosci., 12, 95-110.
Robinson, T. E., Gomy, G., Mitton, E. & Kolb, B. (2001) Cocaine self-administration alters the morphology of dendrites and dendritic spines in the nucleus accumbens and neocortex. Synapse, 39, 257-266.
Rosenblad, C., Georgievska, B. & Kirik, D. (2003) Long-term striatal overexpression of GDNF selectively downregulates tyrosine hydroxylase in the intact nigrostriatal dopamine system. Eur. J. Neurosci., 17, 260-270.
Roth-Deri, I., Zangen, A., Aleli, M., Goelman, R. G., Pelled, G., Nakash, R., Gispan-Herman, I., Green, T., Shaham, Y. & Yadid, G. (2003) Effect of experimenter-delivered and self-administered cocaine on extracellular beta-endorphin levels in the nucleus accumbens. J. Neurochem., 84, 930-938.
Schaar DG, Sieber BA, Dreyfus CF, Black I B. Regional and cell-specific expression of GDNF in rat brain. Exp Neurol. 1993 December;124(2):368-71.
Sklair-Tavron, L., Shi, W. X., Lane, S. B., Harris, H. W., Bunney, B. S. & Nestler, E. J. (1996) Chronic morphine induces visible changes in the morphology of mesolimbic dopamine neurons. Proc. Natl. Acad. Sci. U S. A., 93, 11202-11207.
Smith M A, Makino S, Kvetnansky R, Post R M. Effects of stress on neurotrophic factor expression in the rat brain. Ann N Y Acad. Sci. 1995 Dec. 29;771:234-9.
Snow, D. M., Smith, J. D., Booze, R. M., Welch, M. A. & Mactutus, C. F. (2001) Cocaine decreases cell survival and inhibits neurite extension of rat locus coeruleus neurons. Neurotoxicol. Teratol., 23, 225-234.
Sullivan, A. M., Pohl, J. & Blunt, S. B. (1998) Growth/differentiation factor 5 and glial cell line-derived neurotrophic factor enhance survival and function of dopaminergic grafts in a rat model of Parkinson's disease. Eur. J. Neurosci., 10, 3681-3688.
Suter-Crazzolara C, Unsicker K. GDNF is expressed in two forms in many tissues outside the CNS. Neuroreport. 1994 Dec. 20;5(18):2486-8.
Tornatore, C., Baker-Cairns, B., Yadid, G., Hamilton, R., Meyers, K., Atwood, W., Cummins, A., Tanner, V. & Major, E. (1996) Expression of tyrosine hydroxylase in an immortalized human fetal astrocyte cell line; in vitro characterization and engraftment into the rodent striatum. Cell Transplant, 5, 145-163.
Tomatore, C. S., Bankiewicz, K., Lieberman, D. & Major, E. O. (1993) Implantation and survival of a human fetal astrocyte cell line in the basal ganglia of the non-human primate, rhesus money. (abstract). J. Cell. Biochem., 17E, 227.
Treanor J J, Goodman L, de Sauvage F, Stone D M, Poulsen K T, Beck C D, GrayC, Armanini M P, Pollock R A, Hefti F, Phillips H S, Goddard A, Moore M W, Buj-Bello A, Davies A M, Asai N, Takahashi M, Vandlen R, Henderson CE, Rosenthal A. Characterization of a multicomponent receptor for GDNF. Nature. 1996 Jul. 4;382(6586):80-3
Tresco, P. A., Biran, R. & Noble, M. D. (2000) Cellular transplants as sources for therapeutic agents. Adv. Drug Deliv. Rev., 42, 3-27.
Trupp M, Arenas E, Fainzilber M, Nilsson AS, Sieber BA, Grigoriou M, Kilkenny C, Salazar-Grueso E, Pachnis V, Arumae U. Functional receptor for GDNF encoded by the c-ret proto-oncogene. Nature. 1996 Jun. 27;381(6585):785-9.
Vrana, S. L., Vrana, K. E., Koves, T. R., Smith, J. E. & Dworkin, S. I. (1993) Chronic cocaine administration increases CNS tyrosine hydroxylase enzyme activity and mRNA levels and tryptophan hydroxylase enzyme activity levels. J. Neurochem., 61, 2262-2268.
Yadid, G., Fitoussi, N., Kinor, N., Geffen, R. & Gispan, I. (1999) Astrocyte line SVG-TH grafted in a rat model of Parkinson's disease. Prog. Neurobiol., 59, 635-661.
Zangen, A., Overstreet, D. H. & Yadid, G. (1999) Increased catecholamine levels in specific brain regions of a rat model of depression: normalization by chronic antidepressant treatment. Brain Res., 824, 243-250.

1. A method of attenuating cocaine seeking behavior in a subject, the method comprising administering into a selected region of a brain of the subject a physiologically effective amount of glial cell-derived neurotrophic factor (GDNF) by means of a controlled release mechanism.

2. The method of claim 1, wherein said selected region of a brain includes a NAc/striatal border.

3. The method of claim 1, wherein said physiologically effective amount comprises 1 to 20 μg per subject per day.

4. The method of claim 1, wherein said controlled release mechanism is selected from the group consisting of an implanted population of cells capable of secreting GDNF, a pump capable of releasing GDNF, and a substrate capable of releasing GDNF bound thereto.

5. A pharmaceutical composition, the pharmaceutical composition comprising as an active ingredient a physiologically effective amount of GDNF and physiologically acceptable carriers and excipients;
wherein the pharmaceutical composition is effective in attenuating cocaine-seeking behavior in a subject when administration into a selected region of a brain of said subject is performed.

6. The pharmaceutical composition of claim 5, wherein said administration into said selected region of said brain of said subject includes use of a controlled release mechanism.

7. The pharmaceutical composition of claim 5, wherein said selected region of said brain includes a NAc/striatal border.

8. The pharmaceutical composition of claim 5, wherein said physiologically effective amount comprises 1 to 20 μg per subject per day.

9. The pharmaceutical composition of claim 6, wherein said controlled release mechanism is selected from the group consisting of an implanted population of cells capable of secreting GDNF, a pump capable of releasing GDNF, and a substrate capable of releasing GDNF bound thereto.

10. An article of manufacture comprising:
(a) a pharmaceutical composition comprising as an active ingredient a physiologically effective amount of GDNF and physiologically acceptable carriers and excipients;
(b) packaging material; and
(c) instructions for administration into a selected region of a brain of a subject said pharmaceutical composition as a means of attenuating cocaine-seeking behavior in said subject.

11. The article of manufacture of claim 10, further comprising a controlled release mechanism identified in said instructions as a means of said administration into said selected region of said brain of said subject said physiologically effective amount of GDNF.

12. The article of manufacture of claim 10, wherein said selected region of said brain includes a NAc/striatal border.

13. The article of manufacture of claim 10, wherein said physiologically effective amount comprises 1 to 20 μg per subject per day.

14. The article of manufacture of claim 11, wherein said controlled release mechanism is selected from the group consisting of an implanted population of cells capable of secreting GDNF, a pump capable of releasing GDNF, and a substrate capable of releasing GDNF bound thereto.
